(19) United States
(12) Patent Application Publication
(10) Pub. No.: US 2014/0379299 A1
(43) Pub. Date: Dec. 25, 2014

(54) SYSTEM AND A METHOD TO DETECT HYDROGEN LEAKAGE USING NANO-CRYSTALLIZED PALLADIUM GRATINGS

(71) Applicant: Jawaharial Nehru Centre for Advanced Scientific Research, Bangalore, Karnataka (IN)

(72) Inventors: Giridhar U. Kulkarni, Bangalore (IN); Ritu Gupta, Bangalore (IN); Abhay A. Sagade, Bangalore (IN)

(73) Assignee: Jawaharlal Nehru Centre for Advanced Scientific Research, Bangalore, Karnataka (IN)

(21) Appl. No.: 14/372,693

(22) PCT Filed: Oct. 1, 2012
(86) PCT No.: PCT/IB2012/055245

§ 371 Date: Jul. 16, 2014

(30) Foreign Application Priority Data
Jan. 18, 2012 (IN) 205/CHE/2012

(51) Int. Cl. G01N 21/47; C03C 25/00; G01M 3/38; C03C 25/10; G01N 33/00; G01N 21/85

(52) U.S. Cl. CPC G01N 21/4788; G01N 33/0062; G01N 33/005; G01N 21/85; G01M 3/38; C03C 25/108; C03C 25/002; G01N 2033/0068; G01N 2021/8578
USPC 702/182; 73/23.2; 427/163.2

(57) ABSTRACT

Embodiments of the present disclosure relate to a system and method to detect hydrogen leakage. The system uses a fluid sensing apparatus (104), a light source (120) and a photo detector (122). The nano-crystallized palladium gratings (118) are used as sensors which expand sensitively upon exposure to the hydrogen (H₂). In an embodiment, the hydrogen sensing is based on monitoring the changes in the diffraction efficiency (DE) which is defined as the ratio of the first and the zeroth order diffracted beam intensities. The diffraction efficiency undergoes large and sudden changes as the nano-crystalline Pd grating becomes highly disordered due to PdHx formation. An embodiment of the present disclosure also relates to producing nanocrystalline Pd diffraction gratings along with the design and fabrication aspects of an indigenously built optical diffraction cell for H₂ sensing.

### FIELD OF THE INVENTION

The present disclosure relates to detect hydrogen leakage. More particularly, the embodiments of the present disclosure relates to a system for detecting hydrogen leakage using nano-crystalline Pd grating and a method of performing optical diffraction on the same.

### BACKGROUND

A hydrogen sensing device is used for determining the concentration of hydrogen in a fluid atmosphere. Hydrogen gas has very small molecules making it more prone to leakage than other gases. As hydrogen fluid has no color or odour, and has low viscosity and low molecular weight, it is difficult to detect the hydrogen leakage in a confined space. Additionally, hydrogen upon exposure to air generates fire and the ignition of hydrogen-air mixture is nearly invisible. To detect leakage of hydrogen, many sensors have been developed in the past.

Commercially available sensors can detect the presence of hydrogen and then close valves, shut down equipment, or trigger alarms. However, current technologies typically have limitations related to cost, speed of operation, susceptibility to interference from other gases, and temperature range. The conventional hydrogen (H₂) sensors uses minimum amount of oxygen at the sensor location to detect the concentration of a hydrogen fuel. The oxygen concentration at the sensor location is reduced if the concentration of hydrogen increases. This method generates fire when great amount of hydrogen mixed with air ignites which is an inefficient method to detect the concentration of hydrogen in a particular confined space.

Few conventional techniques for hydrogen sensing use materials that respond to H₂ sensitivity, for example, hydrogen uranyl phosphate, zinc oxide (ZnO) nanorods, platinum (Pt) nanoparticles, tin oxide (SnO₂) coated carbon nanotubes, tungsten nanowires and graphene based materials. Also, Palladium (Pd) based nanomaterials have been investigated extensively due to high hydrogen solubility and favourable reaction kinetics. Pd is so selective to H₂ adsorption that it exhibits extremely low sensitivity to other gases such as carbon monoxide (CO), chloride (Cl₂), sulphur oxide (SO₂), hydrogen sulphide (H₂S), Nitrogen monoxide (NOₓ) and hydrocarbons. Pd undergoes lattice expansion to form Pd hydride reversibly at room temperature. Using this property, a variety of electrical and optical H₂ sensors have been developed so far. The method to detect the hydrogen leakage used electrical device providing electrical contacts to individual nanotubes or nanowires which resulted in more time consumption and cost prohibitive. Also, the usage of electricity in presence of hydrogen is always a matter of concern considering possible arcing.

Few conventional optical H₂ sensors used optical fibre coated with Pd and monitored changes in the path length due to expansion upon Pd hydride formation. Further, several optical sensors based on transmittance were developed for sensing hydrogen. Other optical sensors are based on reflectivity of micromirrors, reflectance and expansion through fibre Bragg gratings and long period gratings, interferometry with optical fibres, surface plasmon resonance and nanoplasmonics etc. which are very complex.

Therefore, there is a need of an improved hydrogen fluid sensing apparatus for detecting hydrogen leakage to overcome the above-mentioned problems.

### SUMMARY

The shortcomings of the prior art are overcome through the provision of a method, an apparatus and a system as described in the description.

The present disclosure provides a system performing optical diffraction to detect hydrogen leakage using nano-crystallised palladium gratings. The system comprises a fluid sensing apparatus, one or more optical sources, one or more photo detectors, a computing device and a storage unit. The fluid sensing apparatus comprises a chamber placed between the one or more optical sources and the one or more photo detectors, an inlet and an outlet connected to the chamber. The chamber comprises a front glass substrate and a rear glass substrate and one or more nano-crystallised palladium gratings. The chamber is connected between the inlet and the outlet through which a predetermined concentration of hydrogen fluid flows in and out of the chamber respectively. The chamber is provisioned with the front glass substrate and the rear glass substrate such that the front glass substrate is facing one or more optical sources and the rear glass substrate is facing one or more photo detectors. Also, the front glass substrate and rear glass substrate are aligned parallel to each other on the chamber. One or more nano-crystallised palladium gratings are fabricated on the rear glass substrate inside the chamber and are facing the front glass substrate. The one or more nano-crystallised palladium gratings expand upon sensing the hydrogen fluid present inside the chamber. The one or more optical sources radiates an optical beam on to the one or more nano-crystallised palladium gratings through the front glass substrate and the radiated optical beam diffracts out from the expanded one or more nano-crystallised palladium gratings through the rear glass substrate. The one or more photo detectors are provided for detecting a diffraction angle of the diffracted optical beam. The one or more optical sources, the front glass substrates, the rear glass substrates, the one or more nano-crystallised palladium gratings and the one or more photo detectors are aligned with each other. The computing device is coupled to the one or more photo detectors for computing a diffraction efficiency of the diffraction angle and for comparing the computed diffraction efficiency with predetermined diffraction efficiency. If a variation of the diffraction efficiency with respect to the predetermined diffraction is noted then hydrogen leakage is detected. The predetermined diffraction efficiency is stored in the storage unit.

An embodiment of the present disclosure discloses a fluid sensing apparatus. The fluid sensing apparatus comprises a chamber, an inlet and an outlet, a front glass substrate and a rear glass substrate and one or more nano-crystallised palladium gratings. The chamber is connected between the inlet and the outlet through which a predetermined concentration of hydrogen fluid flows in and out of the chamber respectively. The front glass substrate and the rear glass substrate are provisioned on the chamber such that they are aligned parallel to each other. The one or more nano-crystallised palladium gratings are fabricated on the rear glass substrate inside the chamber which expands upon sensing the hydrogen fluid.

An embodiment of the present disclosure discloses a method for detecting hydrogen leakage. The method comprises steps of firstly receiving a predetermined concentration of hydrogen fluid by a chamber of a fluid sensing apparatus through an inlet. The chamber is placed between one or more optical sources and one or more photo detectors. One or more nano-crystallised palladium gratings are fabricated inside the chamber on a rear glass substrate which is provisioned on the chamber. The one or more nano-crystallised palladium gratings expand upon sensing the hydrogen fluid present inside the chamber. Secondly, directing an optical beam from the one or more optical sources on to the one or more nano-crystallised palladium gratings through a front glass substrate provisioned on the chamber. The optical beam gets diffracted from the expanded one or more nano-crystallised palladium gratings through the rear glass substrate. The front glass substrate and rear glass substrate are aligned parallel to each other on the chamber. Thirdly, a diffraction angle of the diffracted optical beam is directed by the one or more photo detectors. Fourthly, a diffraction efficiency of the diffraction angle is computed. The computed diffraction efficiency is compared with predetermined diffraction efficiency by a computing device coupled to the one or more photo detectors. If a variation of diffraction efficiency with respect to the predetermined diffraction efficiency is noted then hydrogen leakage is detected.

An embodiment of the present disclosure discloses a method of fabricating one or more nano-crystallised palladium gratings. The method comprises steps of firstly placing a polydimethylsiloxane (PDMS) stamp having a predetermined grating structure on a rear glass substrate. Secondly, a predetermined measurement of toluene solution is dropped at an edge of the PDMS stamp on the rear glass substrate. The toluene solution comprises palladium (Pd) hexadecylthiolate. Thirdly, the PDMS stamp dropped with the toluene solution is annealed at a first predetermined temperature on a hot plate for a predetermined time interval. Fourthly, the annealed PDMS stamp is cooled to a second predetermined temperature. Lastly, the PDMS stamp is removed from the rear glass substrate to form the one or more nano-crystallised palladium gratings.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The features of the present disclosure are set forth with particularity in the appended claims. The disclosure itself, together with further features and attended advantages, will become apparent from consideration of the following detailed description, taken in conjunction with the accompanying drawings. One or more embodiments of the present disclosure are now described, by way of example only, with reference to the accompanied drawings wherein like reference numerals represent like elements and in which:

FIG. 1 illustrates an exemplary system to detect hydrogen leakage according to an embodiment of the present disclosure;

FIG. 2 illustrates a method to detect hydrogen leakage according to an embodiment of the present disclosure;

FIG. 3 illustrates an exemplary fluid sensing apparatus used for detecting hydrogen leakage according to an embodiment of the present disclosure;

FIG. 4a illustrates an exemplary response curve in terms of Diffraction Efficiency (DE) according to an embodiment of the present disclosure;

FIG. 4b illustrates an example showing variation in DE along with a response time with respect to exposure of certain concentration of H₂ according to an embodiment of the present disclosure;

FIGS. 4c and 4d illustrates exemplary Atomic Force Microscopy (AFM) height images of Pd grating before and after exposure to hydrogen (H₂) and nitrogen (N₂) according to an embodiment of the present disclosure;

FIG. 4e illustrates Optical profiler images of Pd gratings according to an embodiment of the present disclosure;

FIG. 4f illustrates schematic showing the working principle of the nano-crystallised palladium gratings according to an embodiment of the present disclosure;

FIGS. 5a and 5b illustrate a method of fabricating one or more nano-crystallised palladium gratings according to an embodiment of the present disclosure;

FIG. 5c illustrates exemplary Scanning Electronic Microscope (SEM) image of the Pd grating with the corresponding Energy Dispersive Detector (EDS) spectrum according to an embodiment of the present disclosure;

FIG. 6a illustrates an exemplary use of multiple fluid sensing apparatus across the hydrogen gas line between production station and hydrogen utility unit according to an embodiment of the present disclosure;

FIG. 6b illustrates an exemplary graph of change in diffraction efficiency (DE) with respect to varying time pulses when H₂ leaks in N₂ pipeline according to an embodiment of the present disclosure; and

FIG. 6c illustrates an exemplary graph between change in diffraction efficiency (DE) and varying time pulses when air leaks in continuous constant H₂ flow according to an embodiment of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

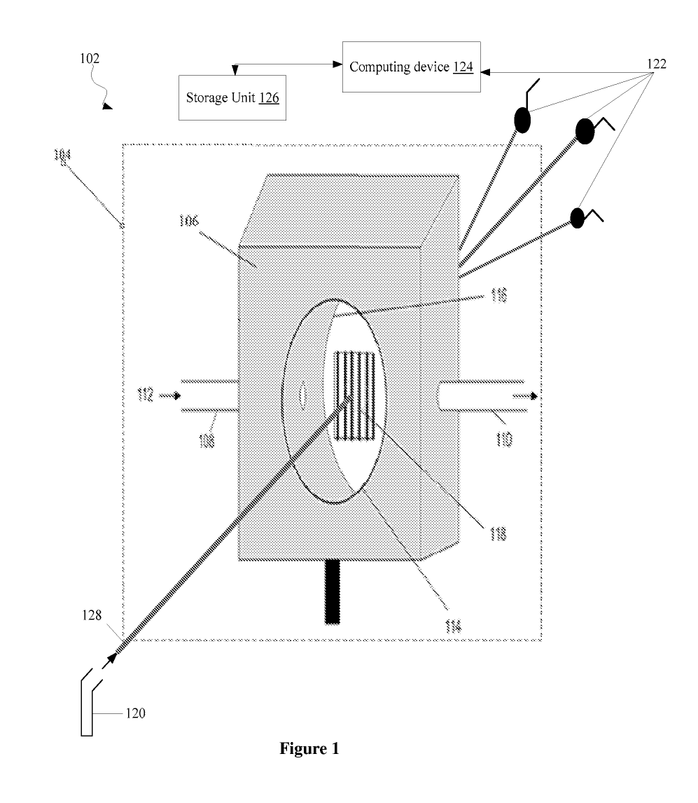

FIG. 1 illustrates an exemplary system to detect hydrogen leakage according to an embodiment of the present disclosure. The system 102 comprises a fluid sensing apparatus 104, one or more optical sources 120, one or more photo detectors 122, a computing device 124 and a storage unit 126. The fluid sensing apparatus 104 comprises a chamber 106 and an inlet 108 and an outlet 110 connected to the chamber 106. The chamber 106 comprises a front glass substrate 114, a rear glass substrate 116 and one or more nano-crystallised palladium gratings 118. The chamber 106 of the fluid sensing apparatus 104 is placed between the optical sources 120 and photo detectors 122. In an embodiment, the optical sources 120 are placed in front of the chamber 106 and photo detectors 122 are placed behind the chamber 106. In an exemplary embodiment, the chamber 106 is made of aluminium. Further, the chamber 106 is connected between the inlet 108 and the outlet 110 through which a predetermined concentration of hydrogen fluid 112 flows in and out of the chamber 106 respectively. In an exemplary embodiment, the hydrogen fluid flows at a flow rate of 10 standard cubic centimetres per minute to 50 standard cubic centimetres per minute. The predetermined concentration of hydrogen fluid 112 is in a range of about 1 percent to about 100 percent. In an embodiment, the hydrogen fluid 112 can be also premixed with nitrogen fluid before passing into the fluid sensing apparatus 104. The front glass substrate 114 and the rear glass substrate 116 are provisioned on the chamber 106 such that the front glass substrate 114 is facing the optical sources 120 and rear glass substrate 116 is facing the photo detectors 122. In an embodiment, the front glass substrate 114 and the rear glass substrate 116 are made of materials including but not limited to a quartz substrate, fiber optics material, transparent plastic, and optical substrates. Further, the front glass substrate 114 and the rear glass substrate 116 are provisioned parallel to each other on the chamber 106 using O-rings. In an exemplary embodiment, the front glass substrate 114 and the rear glass substrate 116 have thickness in a range of about 0.1 millimetres to about 5.0 millimetres. The nano-crystallised palladium gratings 118 are fabricated on the rear glass substrate 116 inside the chamber 106. In an embodiment, the palladium gratings can be fabricated on rigid as well as flexible glass substrates which are transparent. The nano-crystallised palladium gratings 118 are facing the front glass substrate 114 and expand upon sensing the hydrogen fluid 112 present inside the chamber 106. The expansion of the nano-crystallised palladium gratings 118 depends on the concentration of hydrogen fluid 112 inside the chamber 106. The optical sources 120 are used for radiating an optical beam 128 of predetermined wavelength on to the nano-crystallised palladium gratings 118 through the front glass substrate 114. The predetermined wavelength of the optical beam radiated is in a range of about 550 nanometres to about 700 nanometres. In an embodiment, the optical source 120 includes but not limiting to a laser source and light emitting diodes (LED). The radiated optical beam 128 gets diffracted from the expanded nano-crystallised palladium gratings 118 through the rear glass substrate 116. The photo detectors 122 are used for detecting a diffraction angle of the diffracted optical beam. The system 102 of the present disclosure is arranged such that the optical sources 120, the front glass substrate 114, the rear glass substrate 116, the nano-crystallised palladium gratings 118 and the photo detectors 122 are aligned with each other. The computing device 124 is coupled to the photo detectors 122 for computing diffraction efficiency (DE) of the diffraction angle detected by the photo detectors 122 and for comparing the computed diffraction efficiency with predetermined diffraction efficiency. One can use charge coupled device (CCD) camera in place of photo detectors. The diffraction efficiency is ratio of first order to zeroth order diffraction spots intensities. If a variation of the diffraction efficiency with respect to the predetermined diffraction efficiency is noted, then hydrogen leakage is detected. The predetermined diffraction efficiency is a DE value computed in normal condition (i.e. when no leakage, specifically when no hydrogen concentration is present inside the chamber) with pristine palladium grating and with optical beam of particular intensity. The DE value computed is stored in the storage unit 126 associated to the computing device 124.

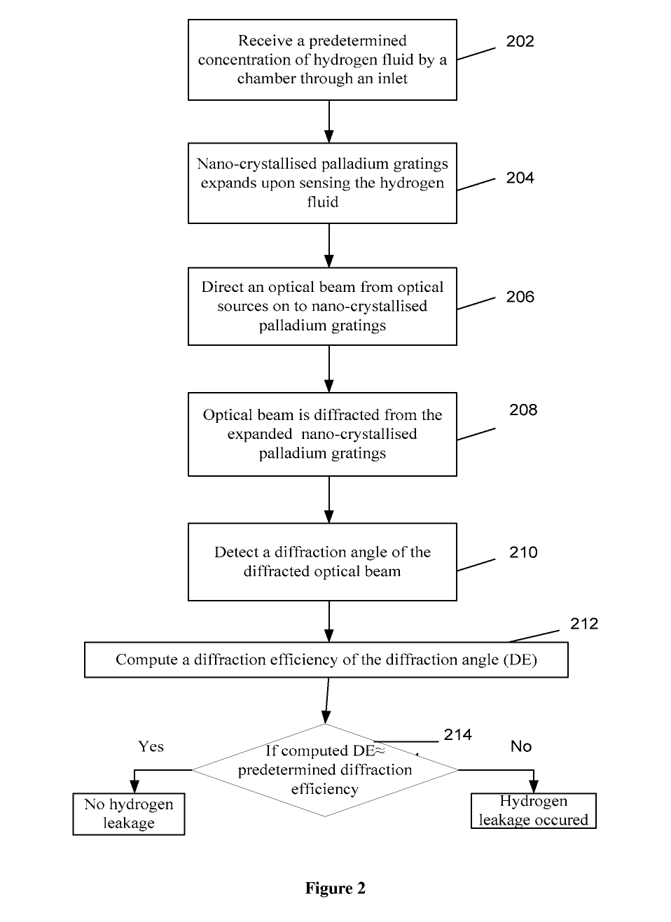

FIG. 2 illustrates a method to detect hydrogen leakage according to an embodiment of the present disclosure. The method comprises steps of receiving a predetermined concentration of hydrogen fluid 112 by a chamber 106 of a fluid sensing apparatus 104 through an inlet 108 at step 202. In an embodiment, the hydrogen fluid flows at flow rate of 10 standard cubic centimetres per minute to 50 standard cubic centimetres per minute. In an exemplary embodiment, the hydrogen fluid 112 can be premixed with nitrogen fluid before passing into the fluid sensing apparatus 104. At step 204, the nano-crystallised palladium gratings 118 fabricated on a rear glass substrate 116 inside the chamber 106 expand upon sensing the hydrogen fluid 112 present inside the chamber 106. The amount of expansion of the nano-crystallised palladium gratings 118 depends on the concentration of hydrogen fluid 112. At step 206, an optical beam 128 from the optical sources 120 is directed on to the nano-crystallised palladium gratings 118 through the front glass substrate 114. At step 208, the radiated optical beam 128 is diffracted from the expanded nano-crystallised palladium gratings 118 through the rear glass substrate 116. A diffraction angle of the diffracted optical beam is detected at step 210 by the photo detectors 122. One can use charge coupled device (CCD) camera in place of photo detectors. At step 212, a computing device 124 coupled to the photo detectors 122 computes a diffraction efficiency of the diffraction angle and the compares the computed diffraction efficiency with the predetermined diffraction efficiency stored in a storage unit 126 coupled to the computing device 124. The diffraction efficiency is a ratio of first order to zeroth order diffraction spots intensities. The comparison of the computed diffraction efficiency with the predetermined diffraction efficiency is performed at step 214. There is no hydrogen leakage if the computed diffraction efficiency is equal or almost equal to the predetermined diffraction efficiency. But, if the computed diffraction efficiency is not equal to the predetermined diffraction efficiency then hydrogen leakage is believed to be occurred. The predetermined diffraction efficiency is a DE value computed in normal condition (i.e. when no leakage, specifically when no hydrogen concentration is present inside the chamber) with pristine palladium grating and with optical beam of particular intensity.

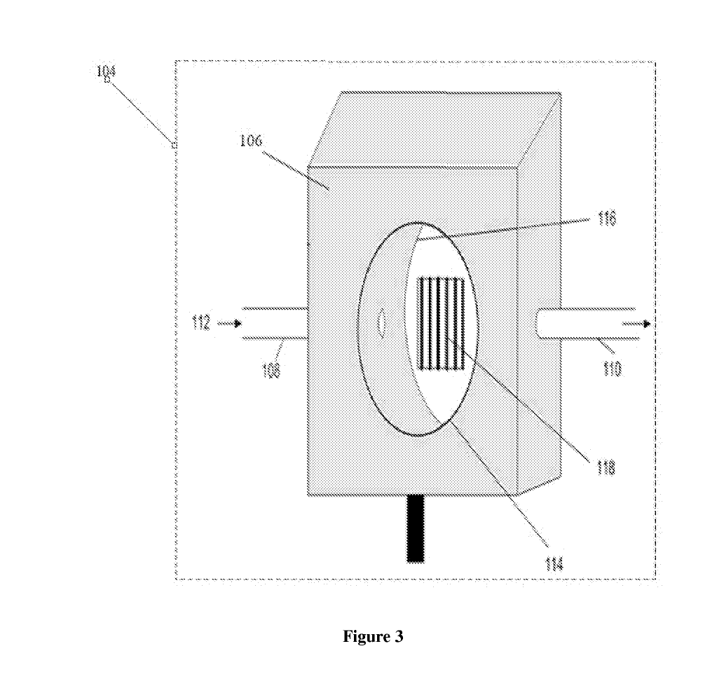

FIG. 3 illustrates an exemplary fluid sensing apparatus 104 used for detecting hydrogen leakage according to an embodiment of the present disclosure. The fluid sensing apparatus 104 comprises a chamber 106 connected between an inlet 108 and an outlet 110 through which a predetermined concentration of hydrogen fluid 112 flows in and out of the chamber 106 respectively. In an embodiment, the hydrogen fluid flows at flow rate of 10 standard cubic centimetres per minute to 50 standard cubic centimetres per minute. A front glass substrate 114 and a rear glass substrate 116 are made of materials including but not limited to a quartz substrate, fiber optics material, transparent plastic and optical substrates. In an embodiment, the front glass substrate 114 and a rear glass substrate 116 and are provisioned on the chamber 106 using at least one of O rings, rubber bellow seal rings, and other related sealing rings. The front glass substrate 114 and rear glass substrate 116 are aligned parallel to each other on the chamber 106. In an embodiment, the chamber 106 is made of aluminium. The front glass substrate and the rear glass substrate have thickness in a range of about 0.1 millimetres to about 5.0 millimetres. The nano-crystallised palladium gratings 118 are fabricated on the rear glass substrate inside the chamber 106 and get expanded upon sensing the hydrogen fluid 112 present inside the chamber 106. In an embodiment, the palladium gratings can be fabricated on rigid and flexible transparent glass substrates. The expansion of the nano-crystallised palladium gratings 118 depends on the concentration of hydrogen fluid 112.

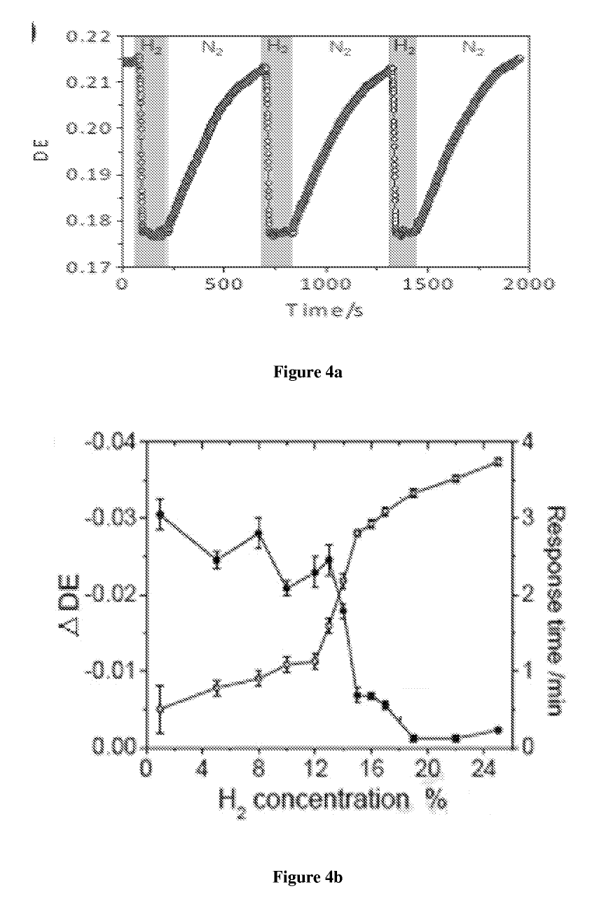

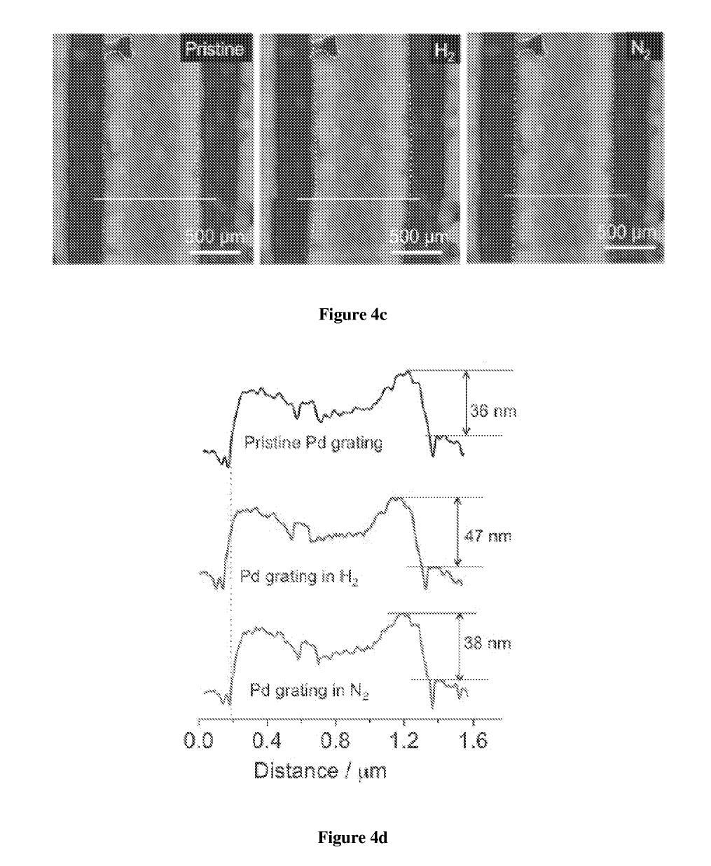

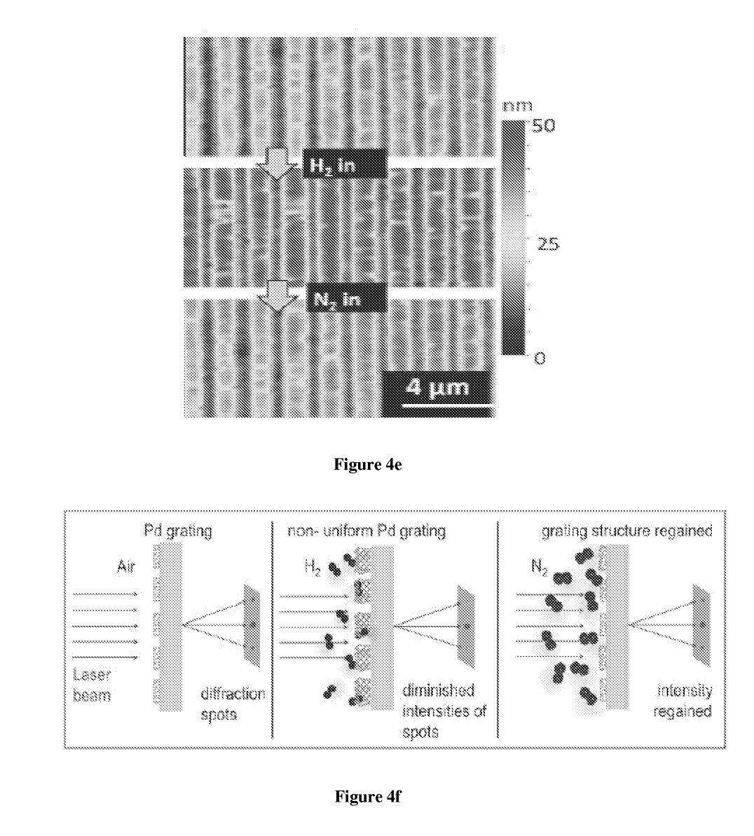

FIG. 4a illustrates an exemplary response curve in terms of Diffraction Efficiency (DE) of the Pd gratings after exposure to hydrogen (H₂) according to an embodiment of the present disclosure. In the illustrated figure, 22% concentration of hydrogen is premixed with Nitrogen (N₂). Upon exposure of nano-crystallised palladium gratings to H₂ (22%, diluted in N₂), the value of DE decreases since the palladium grating responds actively to hydrogen exposure converting Pd to its hydride. As pure N₂ comes in contact, the DE value gradually regains to nearly its original value. The optical observation of Pd hydridation and dehydridation is highly reversible, as shown in three consecutive cycles in FIG. 4a.

FIG. 4b illustrates an example showing variation in DE along with a response time upon exposure to certain concentration of H₂ according to an embodiment of the present disclosure. The change in DE (ΔDE=final value of diffraction efficiency−initial value of diffraction efficiency) and the response time is dependent on H₂ concentration. As H₂ concentration in the mixture increases, for example from 1% to 12%, ΔDE is almost steady. In 15% of H2, the change in ΔDE is sharp and the ΔDE value reaches−0.030, and thereafter increases steadily to a maximum value of 0.037 at 25% H2. At higher concentrations, ΔDE is almost constant. The response is faster for the higher concentrations of hydrogen.

The factors responsible for the observed changes in DE are the changes in optical properties of the grating (refractive index and extinction coefficient, Δη and Δk respectively and the optical density at the given wavelength, OD(λ)) and the grating thickness, t. The mathematical relation that relates these physical quantities to DE is given as:

$$DE = ( \frac{π t}{λ cos θ} ) \{ exp [ \frac{- 2.303 OD ( λ )}{cos θ} ] \} ( Δ {k}^{2} + {Δη}^{2} ) \qquad ( 1 )$$

For example, the Δη and Δk are determined from the difference of refractive index values for Pd (η=1.936 and k=4.38) and the surrounding air (η=1 and k˜0) as medium. Using Δη Pd=0.936, Pd Δk=4.38, OD=0.838, t=40 nm, and θ=25.8° in Equation 1, DE value of 0.505 is estimated for the pristine Pd grating i.e. under non-exposure of any fuel (hydrogen or nitrogen) which is considerably higher than the predetermined experimental value of 0.215. The diminished value is due to the effect of disorder associated with the nano-crystalline nature of the grating lines. The rough edges of the Pd stripes may also affect the DE value.

FIGS. 4c and 4d illustrates exemplary Atomic Force Microscopy (AFM) height images of Pd grating before and after exposure to hydrogen (H₂) and nitrogen (N₂) according to an embodiment of the present disclosure. For example, Pd gratings are pristine before introducing the hydrogen (H₂). After introduction of H₂, the Pd stripes become little uneven as illustrated in FIG. 4c. FIG. 4d illustrates that the height of the Pd stripe is increased noticeably and the Pd stripe swells due to hydride formation upon exposure to H₂. Pd upon hydridation expands upto ˜7.3%, and results in increased thickness and/or height of the grating lines by ˜10 nanometres.

FIG. 4e illustrates optical profiler images of Pd gratings according to an embodiment of the present disclosure. Optical profiler images of Pd grating are illustrated after being introduced to a stream of H₂ gas and after being purged with N₂. The bar shown alongside relates to height variations in a range of about 25 nanometres to 50 nanometres.

FIG. 4f illustrates schematic showing the working principle of the nano-crystallised palladium gratings according to an embodiment of the present disclosure. The change in the morphology of the Pd grating lines influences the diffracted intensities. The pristine Pd grating exhibits uniformly diffractive areas with few spots corresponding to relatively higher thickness. Upon H₂ exposure, the grating image developed spots with lower intensities after diffraction, due to lattice expansion. The change in coloration is quite prominent indicating that the grating lines get swollen and also become defective. Upon exposure to nitrogen, intensity of diffractive spots is regained. The Pd grating expands after sensing hydrogen. Both Δη and Δk decreases upon hydridation, and thus brings down the DE value. The increase in grating thickness “t” due to PdHx formation has an opposing effect and thus causes minimal overall change in DE. But in the nano-crystalline grating sensor the presence of defects and non-uniform expansion of the grating lines actually have adverse effects on diffraction causing DE to decrease rather than increase.

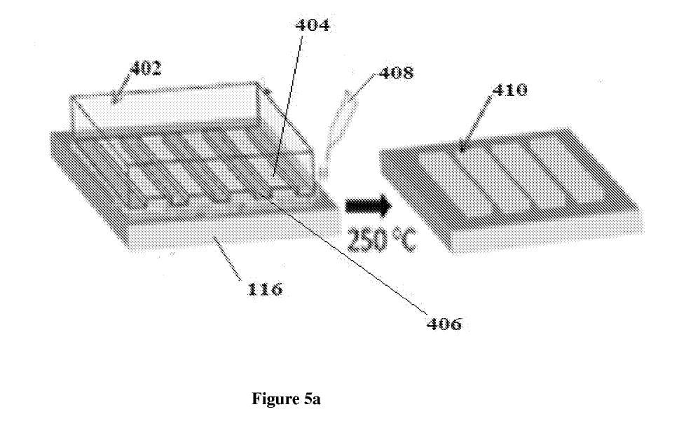

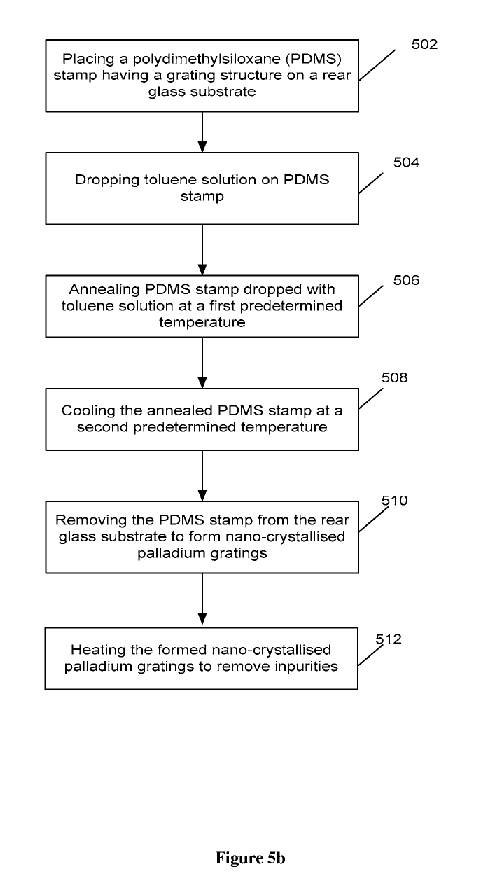

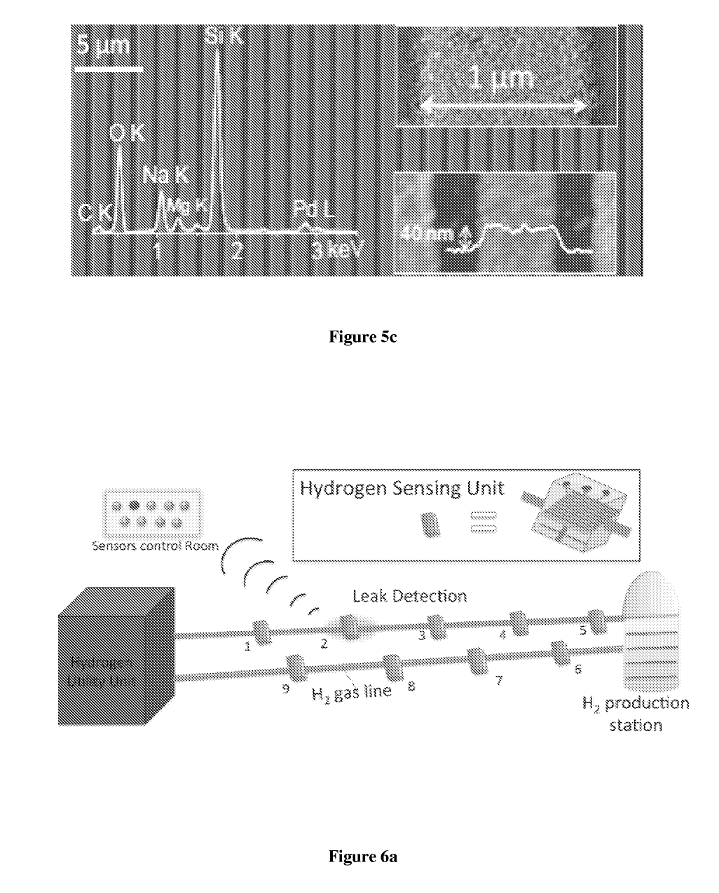

FIGS. 5a and 5b illustrates a method of fabricating nano-crystallised palladium gratings according to an embodiment of the present disclosure. The FIG. 5a shows a polydimethylsiloxane (PDMS) stamp 402 having a predetermined grating structure. The PDMS stamp 402 has a width in a range of about 500 nanometres to about 550 nanometres. The predetermined grating structure comprises pitch 404 having a length in a range of about 1.0 micrometres to about 2.0 micrometres and a thickness in a range of about 0.1 micrometres to about 2.0 micrometres. Each pitch 404 has width in a range of about 0.1 micrometres to about 1.0 micrometres. The grating structure also comprises grooves 406 which define the separation between each pitch 404. The grooves 406 of the grating structure have a depth in a range of about 140 nanometres to about 160 nanometres and a width in a range of about 940 nanometres to about 960 nanometres. A rear glass substrate 116 is shown in the illustrated FIG. 5a. The rear glass substrate 116 is made of at least one of quartz substrate, fiber optics material, transparent plastic and optical substrates. A toluene solution 408 comprising palladium (Pd) hexadecylthiolate is used to form the nano-crystallised palladium gratings.

FIG. 5b illustrates a method of forming nano-crystallised palladium gratings. The method comprises steps of firstly placing the polydimethylsiloxane (PDMS) stamp 402 having the predetermined grating structure on a rear glass substrate 116 at step 502. Secondly, a predetermined measurement of toluene solution 408 comprising palladium (Pd) hexadecylthiolate is dropped at an edge of the PDMS stamp 402 on the rear glass substrate 116 at step 504. The predetermined measurement of the toluene solution 408 is in a range of about 40 micro litres to about 60 micro litres. Next at step 506, the PDMS stamp 402 dropped with the toluene solution 408 is annealed at a first predetermined temperature, in a range of about 200 degrees Celsius to about 300 degrees Celsius. The PDMS stamp 402 is annealed on a hot plate for a predetermined time interval from about 20 minutes to 40 minutes. Later at step 508, the annealed PDMS stamp 402 is cooled to a second predetermined temperature which is room temperature in a range from about 20 degrees Celsius to about 35 degrees Celsius. After cooling, the PDMS stamp 402 is removed from the rear glass substrate 116 to form one or more nano-crystallised palladium gratings at step 510. Further, the nano-crystallised palladium gratings 410 are heated again for about 25 minutes to 35 minutes at temperature in a range 250 degrees Celsius to about 350 degrees Celsius to remove the residual carbon impurities at step 512. The nano-crystallised palladium gratings 410 thus formed has a refractive index in a range of about 0.1 to about 3.0. In an embodiment, the Pd grating parameters can be optimized for maximum sensitivity and can also be fabricated by various lithographic methods.

FIG. 5c illustrates exemplary Scanning Electronic Microscope (SEM) image of the Pd grating with the corresponding Energy Dispersive Spectroscopy (EDS) spectrum according to an embodiment of the present disclosure. For example, the SEM image of the obtained Pd stripes shows a width of ˜1 μm (width of pitch 404) with separation of ˜0.5 μm between each grating. Energy dispersive spectroscopy (EDS) on the pattern showed the presence of Pd with negligible traces of carbon (C) and sulphur (S). The top inset of the FIG. 5c shows the morphology of interconnected and densely packed Pd nanoparticles of ˜5 nm diameter confined to a ˜1 μm wide line as an example. The AFM image in the bottom inset shows the thickness of the line grating to be, for example, ˜40 nm with a roughness of ˜5 nm.

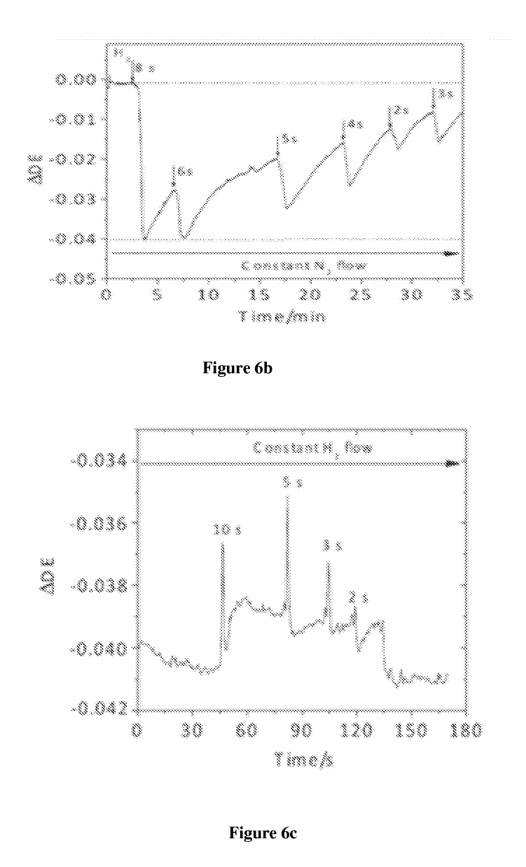

FIG. 6a illustrates an exemplary use of multiple fluid sensing apparatus across the hydrogen gas line between production station and hydrogen utility unit according to an embodiment of the present disclosure. As an example, hydrogen leak takes place near hydro cracking reactors and gas separators which typically work at high pressures or near deformed flanges or at fractured gas lines which typically run for miles. Such a sudden accidental situation leads to a rapid release of H₂. Therefore, on-site detectors are expected to be sensitive and working in proximity to a leak over a wide range of concentrations unlike sensors that work at distance such as Ultrasonic detectors. For example, a large number of fluid sensing apparatus 104 (numbered as 1, 2, 3, to 9) are installed at intervals on a H₂ gas line. The leak detection can be performed easily without any electrical interconnects but by collecting optical signals through optical fibre based communication.

FIG. 6b illustrates exemplary diffraction efficiency (DE) upon leak detection of H₂ in N₂ pipeline according to an embodiment of the present disclosure. In the H₂ leak test, for example, H₂ (25%) is injected as short pulses in continuously flowing N₂ across the grating chamber. With the first pulse of 8 s, the DE decreased steeply to 0.04 and gradually regained as pure N₂ continued to flow. Shorter duration pulses, however, produced corresponding changes in the DE value with the shortest pulse being 1 s.

FIG. 6c illustrates exemplary diffraction efficiency upon air leak detection in continuous constant H₂ flow according to an embodiment of the present disclosure. Air leak is another situation encountered during hydrogen production and transport. For example, the introduction of air in short pulses produced sharp jumps in DE, down to a 0.5 s pulse. Brief exposure to oxygen (in air pulse) inhibits the formation of PdHx instantly but temporarily, giving rise to sharp features.

The present embodiment performs optical diffraction for sensing the hydrogen leakage which is free of complicated and expensive lithography steps.

The present embodiment uses nano-crystallised palladium gratings which works efficiently at room temperature upon exposure to hydrogen (H₂), and is low cost sensing material.

The response time of sensing the leakage is few seconds even with low flow rate of hydrogen fluid (for example, from 10 sccm to 50 sccm). The structural changes such as increase in roughness and defects upon hydridation has an overwhelming but adverse effect on diffraction, perhaps more than the changes in absorptivity and refractive index could bring about.

The effects of sensing the leakage could be repeated over many cycles of operation. In an embodiment, the leakage detected is transmitted through optical communication. The fluid sensing apparatus is portable and inexpensive and consumes very low power.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERENCE TABLE

| Reference Numerals | Description |
|---|---|
| System | 102 |
| Fluid sensing apparatus | 104 |
| Chamber | 106 |
| Inlet | 108 |
| Outlet | 110 |
| Hydrogen fluid | 112 |
| Front glass substrate | 114 |
| Rear glass substrate | 116 |
| Nano-crystallised palladium gratings | 118 |
| Light source | 120 |
| Photo detectors | 122 |
| Computing device | 124 |
| Storage unit | 126 |
| Radiating beam | 128 |
| Polydimethylsiloxane (PDMS) stamp | 402 |
| Pitch | 404 |
| Grooves | 406 |
| Toluene solution | 408 |
| nano-crystallised palladium gratings | 410 |

1. A system to detect hydrogen leakage, said system comprising:
a fluid sensing apparatus comprising a chamber placed between one or more optical sources and one or more photo detectors, said fluid sensing apparatus comprising:
an inlet and an outlet connected to the chamber through which a predetermined concentration of hydrogen fluid flows in and out of the chamber respectively;
a front glass substrate provisioned on the chamber, said front glass substrate is facing one or more optical sources;
a rear glass substrate provisioned on the chamber, said rear glass substrate is facing one or more photo detectors;
one or more nano-crystallized palladium gratings fabricated inside the chamber on the rear glass substrate, said one or more nano-crystallized palladium gratings are facing the front glass substrate, wherein the one or more nano-crystallized palladium gratings expands upon sensing the hydrogen fluid;
the one or more optical sources for radiating an optical beam on to the one or more nano-crystallized palladium gratings through the front glass substrate, wherein the radiated optical beam is diffracted from the expanded one or more nano-crystallized palladium gratings;
the one or more photo detectors for detecting a diffraction angle of the optical beam diffracted from the expanded one or more nano-crystallized palladium gratings through the rear glass substrate; and
a computing device coupled to the one or more photo detectors for computing a diffraction efficiency of the diffraction angle and for comparing the computed diffraction efficiency with a predetermined diffraction efficiency to detect the hydrogen leakage;
wherein the one or more optical sources, the front glass substrates, the rear glass substrates, the one or more nano-crystallized palladium gratings and the one or more photo detectors are aligned with each other.

2. The system as claimed in claim 1, wherein the chamber is made of aluminium.

3. The system as claimed in claim 1, wherein the predetermined concentration of the hydrogen fluid is in a range of about 1 percent to about 100 percent.

4. The system as claimed in claim 1, wherein the hydrogen fluid can be premixed with nitrogen fluid before passing into the fluid sensing apparatus.

5. The system as claimed in claim 1, wherein the front glass substrate and the rear glass substrate are made of a quartz substrate.

6. The system as claimed in claim 1, wherein the front glass substrate and the rear glass substrate are provisioned parallel to each other on the chamber of the fluid sensing apparatus.

7. The system as claimed in claim 1, wherein the front glass substrate and the rear glass substrate has thickness in a range of about 0.1 mm to about 5.0 mm.

8. The system as claimed in claim 1, wherein the front glass substrate and the rear substrate are provisioned on the chamber of the fluid sensing apparatus using O rings.

9. The system as claimed in claim 1, wherein the predetermined diffraction efficiency is stored in a storage unit associated to the computing device.

10. A fluid sensing apparatus comprising:
a chamber connected between an inlet and an outlet through which a predetermined concentration of hydrogen fluid flows in and out of the chamber respectively;
a front glass substrate and a rear glass substrate provisioned on the chamber, said front glass substrate and rear glass substrate are aligned parallel to each other on the chamber; and
one or more nano-crystallized palladium gratings fabricated inside the chamber on the rear glass substrate, said one or more nano-crystallized palladium gratings expands upon sensing the hydrogen fluid.

11. The fluid sensing apparatus as claimed in claim 10, wherein the front glass substrate and the rear glass substrate are provisioned on the chamber using O rings.

12. The fluid sensing apparatus as claimed in claim 10, wherein the front glass substrate and the rear glass substrate are made of a quartz substrate.

13. The fluid sensing apparatus as claimed in claim 10, wherein the front glass substrate and the rear glass substrate have thickness in a range of about 0.1 mm to about 5.0 mm.

14. A method of detecting hydrogen leakage, said method comprising steps of:
receiving a predetermined concentration of hydrogen fluid by a chamber of a fluid sensing apparatus through an inlet, said chamber is placed between one or more optical sources and one or more photo detectors, wherein one or more nano-crystallized palladium gratings are fabricated inside the chamber on a rear glass substrate provisioned on the chamber, said one or more nano-crystallized palladium gratings expands upon sensing the hydrogen fluid;
directing an optical beam from the one or more optical sources on to the one or more nano-crystallized palladium gratings through a front glass substrate provisioned on the chamber, said optical beam is diffracted from the expanded one or more nano-crystallized palladium gratings through the rear glass substrate;
detecting a diffraction angle of the diffracted optical beam by the one or more photo detectors; and
computing a diffraction efficiency of the diffraction angle and comparing the computed diffraction efficiency with a predetermined diffraction efficiency by a computing device coupled to the one or more photo detectors to detect the hydrogen leakage.

15. The method as claimed in claim 14, wherein the predetermined diffraction efficiency is stored in a storage unit coupled to the computing device.

16. The method as claimed in claim 14, wherein the hydrogen fluid can be premixed with nitrogen fluid before passing into the fluid sensing apparatus.

17. A method of fabricating one or more nano-crystallized palladium gratings, said method comprising steps of:
placing a polydimethylsiloxane (PDMS) stamp having a predetermined grating structure on a rear glass substrate;
dropping a predetermined measurement of toluene solution comprising palladium (Pd) hexadecylthiolate at an edge of the PDMS stamp on the rear glass substrate; and
annealing the PDMS stamp dropped with the toluene solution at a first predetermined temperature on a hot plate for a predetermined time interval;
cooling the annealed PDMS stamp to a second predetermined temperature; and
removing the PDMS stamp from the rear glass substrate to form the one or more nano-crystallized palladium gratings.

18. The method as claimed in claim 17, wherein the rear glass substrate is made of a quartz substrate.

19. The method as claimed in claim 17, wherein the PDMS stamp has a width in a range of about 500 nm to about 550 nm.

20. The method as claimed in claim 17, wherein the predetermined measurement of the toluene solution is in a range of about 40 μl to about 60 μl.

21. The method as claimed in claim 17, wherein the predetermined grating structure comprises pitch having a length in a range of about 1.0 μm to about 2.0 μm with grooves having a depth in a range of about 140 nm to about 160 nm.

22. The method as claimed in claim 17, wherein a width of pitch is in a range of about 0.1 μm to about 2.0 μm.

23. The method as claimed in claim 17, wherein the first predetermined temperature is in a range of about 200 degrees Celsius to about 300 degrees Celsius and the second predetermined temperature is a room temperature in a range of about 20 degrees Celsius to about 35 degrees Celsius.

24. The method as claimed in claim 17, wherein the predetermined time interval is in a range of about 20 minutes to 40 minutes.

25. The method as claimed in claim 17 further comprising heating the formed one or more palladium grating in a range of about 250 degrees Celsius to about 350 degrees Celsius for about 25 minutes to 35 minutes.

26. The method as claimed in claim 17, wherein the one or more nano-crystallized palladium gratings has a refractive index in a range of about 0.1 to about 3.0.

27. The method as claimed in claim 21, wherein the grooves of the predetermined gratings structure has a width in a range of about 940 nm to about 960 nm.
